(19) United States
(12) Patent Application Publication
(10) Pub. No.: US 2007/0142738 A1
(43) Pub. Date: Jun. 21, 2007

(54) Dynamic heart rate monitor

(76) Inventor: Chin-Yeh Hung, Chang Pin Town (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/304,560

(22) Filed: Dec. 16, 2005

(51) Int. Cl. A61B 5/04

(52) U.S. Cl. 600/519

(57) ABSTRACT

A dynamic heart rate monitor is disclosed to include a neckband carrying a first electrode set for disposing on the user's neck, a heart rate recording and display unit, a first pair of electric wires fastened to the neckband and coupled between the first electrode set and the heart rate recording and display unit for suspending the heart rate recording and display unit in front of the user's chest, and a second electrode set form of two electrically conductive rubber vacuum mounts and connected to the heart rate recording and display unit through a second pair of electric wires for attaching to the left and right sides of the user's chest to measure the heart rate and to let the measured heart rate voltage signal to be transmitted to the heart rate recording and display unit for output through a LCD in the heart rate recording and display unit or storage in an external computer via a USB connector at the heart rate recording and display unit.

### BACKGROUND OF THE INVENTION

1. Field of the Invention

The present invention relates generally to a hearty rate monitor and more particularly, to a dynamic heart rate monitor, which is made in the form of a necklace, having two electrically conductive rubber vacuum mounts for attaching to the chest to measure the heart rate.

2. Description of the Related Art

The heart rate of a person changes significantly before and after exercise. It is important to monitor the change of heart rate during exercise. In order to obtain the maximum benefit from exercise, the pulse of the heart must be kept at about 60% of the maximum pulse rate. It is harmful to keep exercising when the heart rate is at the maximum level. Therefore, using a heart rate monitor that can conveniently and accurately measure the heart rate during exercise is important.

U.S. Pat. No. 6,533,247 discloses an electrode belt of heart rate monitor. According to this design, the electrode belt comprises an electronic device 70 for measuring the heart rate on the chest, and a belt 71 for securing the electronic device 70 to the chest. The measured signal is then transmitted from the electronic device 70 to a receiver 80 that is fastened to the user's wrist.

This design still has numerous drawbacks as follows:

1. Because electronic device 70 is secured to the user's chest by the belt 71, the binding pressure of the belt 71 causes the user to feel uncomfortable and difficult to breathe during exercise.

2. Because a male has a big breast profile, it is difficult to keep the electronic device 70 in close contact with the chest to measure the heart rate accurately, resulting in a low accuracy.

3. When performing a running exercise, the user's hands are alternatively moved forwards and backwards, resulting in an error in the signal transmitted by the electronic device 70 and received by the receiver 80.

### SUMMARY OF THE INVENTION

The present invention has been accomplished under the circumstances in view. According to one aspect of the present invention, the dynamic heart rate monitor comprises a neckband carrying a first electrode set for disposing on the skin of the user's neck corresponding to the artery at the neck, and a second electrode set formed of two electrically conductive rubber vacuum mounts for adhering to the left and right sides of the user's chest to measure the user's heart rate comfortably.

According to another aspect of the present invention, the two electrically conductive rubber vacuum mounts can be conveniently positively adhered to the left and right sides of a female user's chest to accurately measure the female user's heart rate.

According to still another aspect of the present invention, the first electrode set and the second electrode set of the dynamic heart rate monitor measure the variation of the voltage frequency induced subject to the user's heart rate during exercise, enabling the variation of the voltage frequency to be further calculated into a heart rate data signal for output through a LCD.

### BRIEF DESCRIPTION OF THE DRAWINGS

FIG. 1 is a schematic drawing showing an application example of an electrode belt of heart rate monitor according to U.S. Pat. No. 6,553,247.

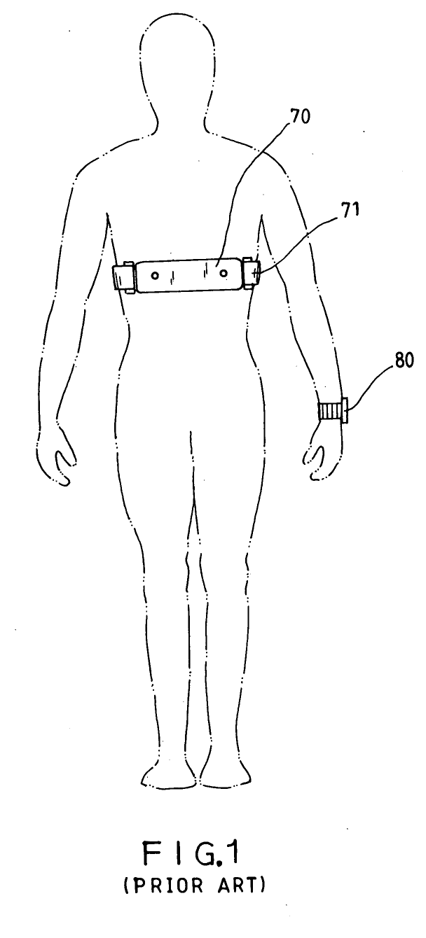

FIG. 2 is a perspective view of a dynamic heart rate monitor according to the present invention.

FIG. 3 is an exploded view of a part of the present invention, showing the relationship between one electrically conductive rubber vacuum mount of the second electrode set and the respective electric wire of the second pair of electric wires.

FIG. 4 is a schematic drawing showing an application example of the dynamic heart rate monitor according to the present invention.

FIG. 5 is a circuit diagram of the dynamic heart rate monitor according to the present invention.

### DETAILED DESCRIPTION OF THE INVENTION

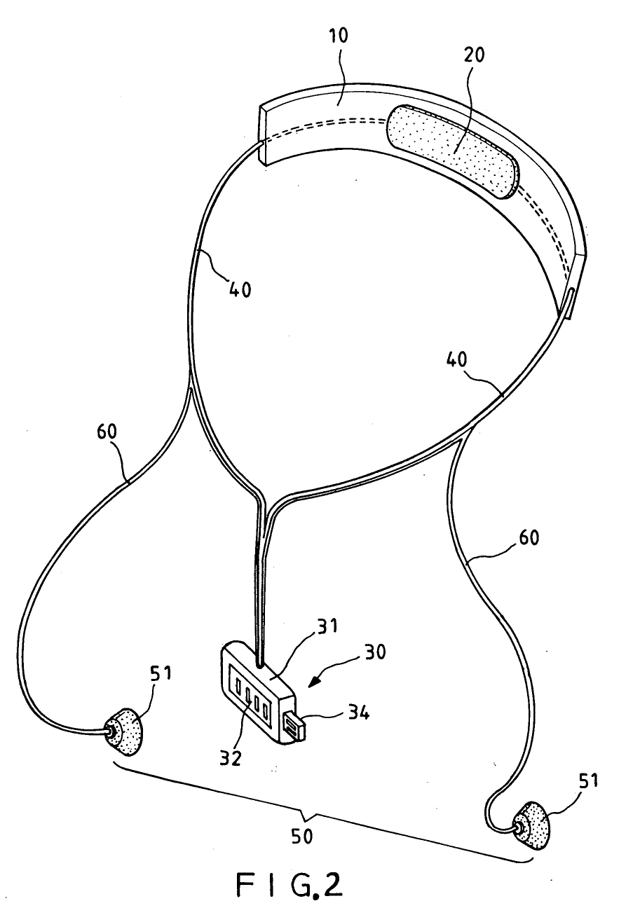

Referring to FIGS. 2 through 5, a dynamic heart rate monitor in accordance with the present invention is shown comprising a neckband 10, a first electrode set 20, a heart rate recording and display unit 30, a first pair of electric wires 40, a second electrode set 50, and a second pair of electric wires 60.

The neckband 10 is a smoothly arched band made of elastic or resilient material and can be clamped on the user's neck comfortably without giving much pressure to the user's neck.

The first electrode set 20 is made out of a conducting rubber or in the form of a conducting pad and mounted in the neck band 10 and curved smoothly outwards from the inner side of the neckband 10 for disposing on the skin of the user's neck closely;

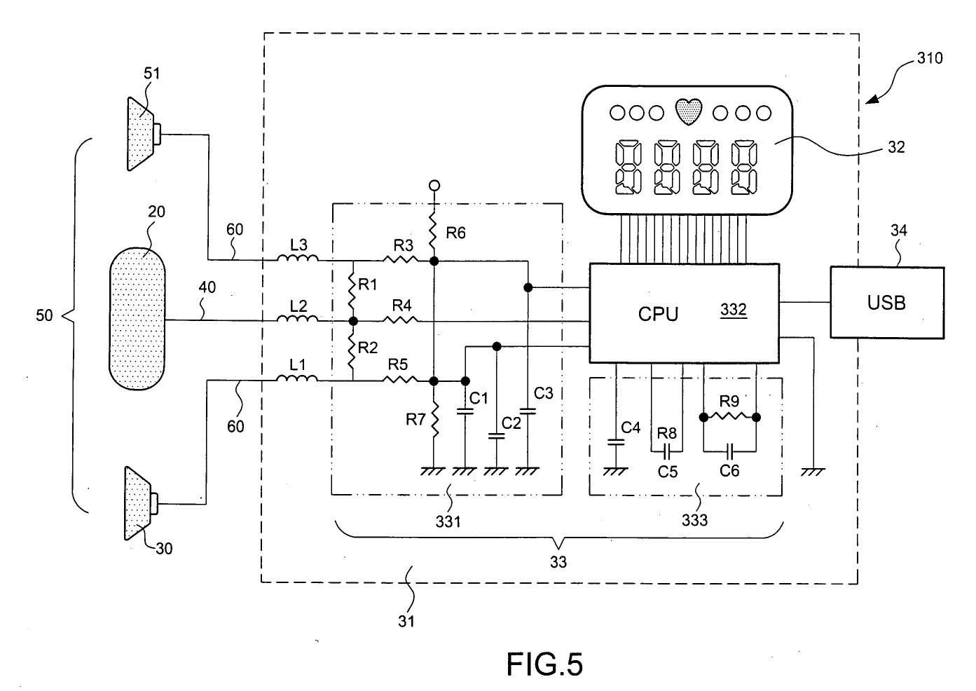

The heart rate recording and display unit 30, as shown in FIGS. 2 and 5, comprises a casing 31, a LCD (Liquid Crystal Display) 32 mounted in the outside wall of the casing 31, a piezoelectric detection circuit 33 mounted inside the casing 31, and a USB (Universal Serial Bus) connector 34 electrically coupled to the piezoelectric detection circuit 33 and extended out of the casing 3;

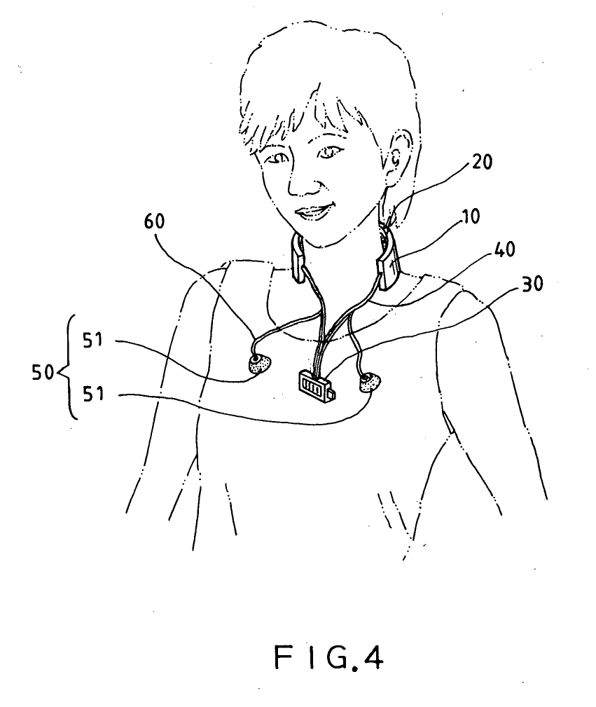

The first pair of electric wires 40 are respectively electrically coupled to the first electrode 20 and respectively extending out of the two distal ends of the neckband 10 and then respectively electrically coupled to the heart rate recording and display unit 30. As shown in FIG. 4, the heart rate recording and display unit 20 forms with the first pair of electric wires 40 and the neckband 10 a necklace for hanging on the neck.

Because the heart rate recording and display unit 20 has a size as small as a pendent or mini MP3 player, it can be inserted into the pocket in front of the chest or kept inside the clothes during exercise.

The second electrode set 50 comprises left and right electrically conductive rubber vacuum mounts 51. The left and right electrically conductive rubber vacuum mounts 51 are made out of a conducting rubber for the advantages of good electrical conductivity and skin friendly characteristic. The left and right electrically conductive rubber vacuum mounts 51 of the second electrode set 50 can easily be adhered to the left and right sides of the user's chest without a binding belt. To a female user, the second electrode set 50 can accurately be adhered to the chest to detect the heart rate accurately.

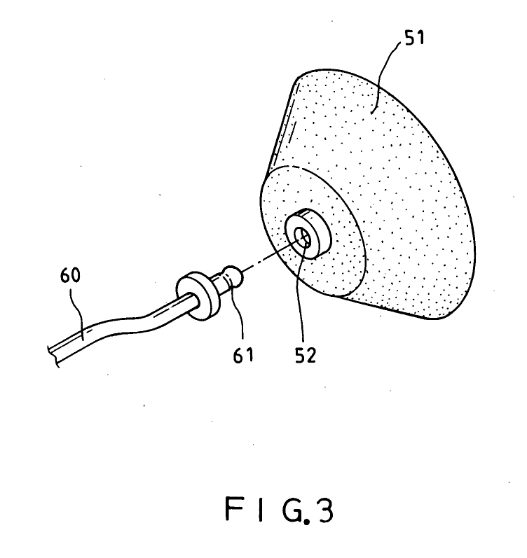

Referring to FIG. 3, each electrically conductive rubber vacuum mount 51 has a socket 52 at the back side. The second pair of electric wires 60 each have one end respectively electrically connected to the heart rate recording and display unit 30 and the other end terminating in an electric plug 61. The electric plugs 61 of the second pair of electric wires 60 are respectively detachably connected to the sockets 52 of the electrically conductive rubber vacuum mounts 51. According to this embodiment, the electrically conductive rubber vacuum mounts 51 are detachably connected to the second pair of electric wires 60. Alternatively, the electrically conductive rubber vacuum mounts 51 can be directly fixedly connected to the second pair of electric wires 60.

FIG. 4 shows an application example of the present invention. As illustrated, the neckband 10 is hung on the user's neck, the first electrode set 20 disposed on the skin of the user's neck corresponding to the artery at the neck, the two electrically conductive rubber vacuum mounts 51 are respectively received inside the user's clothes and adhered to the left and right sides of the chest, and the heart rate recording and display unit 30 is suspending in front of the user's chest.

Referring to FIG. 5, the first electrode set 20 and the second electrode set 50 are respectively electrically coupled to the piezoelectric detection circuit 33 of the heart rate recording and display unit 30. Because the first electrode set 20 is disposed on the skin of the user's neck corresponding to the artery at the neck and the second electrode set 50 is attached to the user's chest, the user's body and the two electrodes 20 and 30 form a close circuit. During circulation of the flood, a voltage is produced. When the heart rate is changed, the frequency of the voltage being monitored is relatively changed. The detected voltage frequency is filtered through inductances L1˜L3 and then processed through a filter rectifier circuit 331 to obtain a signal, which is then sent to a CPU 332 for calculation, and the calculated heart rate data signal is then amplified by an amplifier circuit 333, which can be built in the CPU 332 and externally connected to the CPU 332, allowing the amplified heart rate data signal to be further sent to the LCD 32 for output.

Further, the detected heart rate data that is stored in the CPU 332 can be transmitted to an external PC (Personal Computer) through the USB connector 34 for physical condition analysis by a computer software in the external PC.

As stated above, the invention provides a dynamic heart rate monitor, which uses two electrode sets 20 and 50 to detect the user's heart rate, a neckband 10 to hang the dynamic heart rate monitor on the user's neck. The use of the dynamic heart rate monitor does not interfere with the user's breathing action. The first and second pairs of electric wires 40 and 60 are adapted to transmit the detected heart rate signal to the heart rate recording and display unit 30 for display. Further, the heart rate record can be transmitted to an external computer through the IUSB connector 34 for storage and further physical condition analysis.

A prototype of dynamic heart rate monitor has been constructed with the features of FIGS. 2˜5. The dynamic heart rate monitor functions smoothly to provide all of the features discussed earlier.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A dynamic heart rate monitor comprising:
a neckband, said neckband having a smoothly arched profile for hanging on the user's neck;
a first electrode set carried in said neckband for disposing on the skin of the user's neck;
a heart rate recording and display unit, said heart rate recording and display unit comprising a casing, a LCD (Liquid Crystal Display) mounted in an outside wall of said casing, a piezoelectric detection circuit mounted inside said casing, and a USB (Universal Serial Bus) connector electrically coupled to said piezoelectric detection circuit and extended out of said casing;
a first pair of electric wires respectively electrically coupled to said first electrode and respectively extending out of two distal ends of said neckband and respectively electrically coupled to said heart rate recording and display unit;
a second electrode set, said second electrode set comprising a left electrically conductive rubber vacuum mount and a right electrically conductive rubber vacuum mount respectively attachable to left and right sides of the user's chest by means of a vacuum suction force;
a second pair of electric wires that couple said left and right electrically conductive rubber vacuum mounts to said heart rate recording and display unit;
wherein said first electrode set and said second electrode set measure the heart rate of the user to provide a voltage signal to said piezoelectric detection circuit of said heart rate recording and display unit for processing; said piezoelectric detection circuit of said heart rate recording and display unit comprises a series of inductances adapted to filter the voltage signal received from said first and second electrode sets through said first and second pairs of electric wires, a filter rectifier circuit electrically coupled to said series of inductances and adapted to rectify the waveform of the filtered heart rate voltage signal from said series of inductances, a CPU (Central Processing Unit) electrically coupled to said filter rectifier and adapted to process said heart rate voltage signal into a heart rate data signal and to amplify the heart rate data signal through an amplifier thereof for enabling the amplified heart rate data signal to be sent to said LCD for display and sent to an external computer through said USB connector for storage and analysis.

2. The dynamic heart rate monitor as claimed in claim 1, wherein said neckband is made out of an elastic material.

3. The dynamic heart rate monitor as claimed in claim 1, wherein said first electrode set is made out of an electrically conductive rubber and protrudes over an inner surface of said neckband for disposing on the skin of the user's neck.

4. The dynamic heart rate monitor as claimed in claim 1, wherein said left and right electrically conductive rubber vacuum mounts of said second electrode set each comprise a socket provided at a back side thereof; said second pair of electric wires each have first end respectively electrically coupled to said heart rate recording and display unit and a second end terminating a plug respectively electrically connectable to the sockets of said left and right electrically conductive rubber vacuum mounts of said second electrode set.

5. The dynamic heart rate monitor as claimed in claim 1, wherein said heart rate recording and display unit forms with said first pair of electric wires and said neckband a necklace for hanging on the user's neck.
